(19) United States
(12) Patent Application Publication
(10) Pub. No.: US 2015/0148654 A1
(43) Pub. Date: May 28, 2015

(54) SYSTEM, METHOD AND COMPUTER-ACCESSIBLE MEDIUM FOR PROVIDING AND/OR UTILIZING OPTICAL COHERENCE TOMOGRAPHIC VIBROGRAPHY

(71) Applicant: The General Hospital Corporation, Boston, MA (US)

(72) Inventors: Chang Whanwook, Cambridge, MA (US); Seok-Hyun Yun, Belmont, MA (US)

(21) Appl. No.: 14/411,608

(22) PCT Filed: Jun. 27, 2013
(86) PCT No.: PCT/US2013/048192

§ 371 Date: Dec. 29, 2014

Related U.S. Application Data
(60) Provisional application No. 61/666,212, filed on Jun. 29, 2012.

(51) Int. Cl. A61B 5/00; G01B 9/02; G01N 21/17

(52) U.S. Cl. CPC A61B 5/0095; G01N 21/1702; G01B 9/02091; G01N 2201/06113
USPC 600/407

(57) ABSTRACT

Exemplary embodiments of apparatus, method and computer-accessible medium can be provided for obtaining image information regarding at least one portion of at least one sample. For example, using such exemplary embodiments, it is possible to use at least one arrangement to (i) receive or generate first data regarding a controlled physical excitation of the portion(s) and optical coherence second data associated with the sample(s). Further, it is possible, e.g., using such arrangement(s), to generate the image information based on the first data and the second data, wherein the controlled physical excitation is caused by a non-biological arrangement. The image information can include depth information within the portion(s).

### CROSS REFERENCE TO RELATED APPLICATION(S)

The present application relates to and claims priority from U.S. Provisional Patent Application Ser. No. 61/666,212 filed Jun. 29, 2012, the disclosure of which is incorporated herein by reference in its entirety.

### STATEMENT OF FEDERAL SUPPORT

The present disclosure was made with U.S. Government support under grant number P41RR032042 from National Institute of Health. Thus, the Government has certain rights to the disclosure described and claimed herein.

### FIELD OF THE DISCLOSURE

Exemplary embodiments of the present disclosure relate to optical coherence tomographic vibrography procedure(s), and more particularly to exemplary system, method and computer-accessible medium for capturing vibration snapshots of rapid (e.g., up to GHz) small-scale (e.g., less than about 10 microns) periodic motion with optical coherence tomography for applications including but not limited to middle ear mechanics, otology, biomechanics of cornea and crystalline lens, and rheometry.

### BACKGROUND INFORMATION

Pressure-driven oscillations with nanometer-scale amplitudes at acoustic frequencies can be found in a variety of physical and biological measurement systems. For instance, miniature resonators have been used for radiation pressure cooling [see Ref. 1], sensing molecules [see Ref. 2] and high-precision weighing of single cells [see Ref. 3]. Moreover, acoustic vibrations have been used for dynamic mechanical analysis and rheology as well as photoacoustic imaging and elastography [see Ref. 4]. In such systems, some measurements of the acoustic motion on the surface or within the object under test can be important. While identifying the intrinsic parameters, such as the resonance frequency, may be sufficient for some cases, many applications can require the actual amplitude and phase information and can greatly benefit from a volumetric imaging technique capable of providing spatial graphs of the sample vibration.

Optical interferometry is well suited for a precise measurement of oscillatory motion. Laser Doppler velocimetry and stroboscopic holography have been used for measuring sub-micron-scale vibrations at frequencies up to MHz [see Ref. 5]. However, these techniques can be limited to surface measurements, while optical coherence tomography (“OCT”), an optical analog of ultrasound, offers the potential for capturing motion at various depths in layered or homogeneous samples. Phase-sensitive OCT with sub-nanometer amplitude sensitivity has been used for elastography [see Refs. 6-8], vibration-amplitude mapping [see Ref. 9], and phase microscopy of static or slowly moving samples [see Refs. 10, 11]. In various fields of medicine including ophthalmology and cardiology, OCT have been widely recognized and adapted in medical imaging for disease diagnosis at high spatial resolutions of about 1-15 μm in axial dimension and of 1-20 μm in lateral dimensions [see Refs. 12-15]. With- recent improvement of axial-line (A-line) acquisition rates, applications of OCT continue to expand. Up to a few MHz of A-line acquisition rate was demonstrated with swept source OCT [see Ref. 16], and up to few MHz was achieved [see Ref. 17]. The A-line rates translate to an impressive frame rate of about 1 kHz with about 400 to 2,000 A-lines per frame. However, even with the record A-line rate, samples that move faster than the OCT frame rate can cause undesired image artifacts and make accurate image acquisition and visualization impossible. Therefore, existing applications of OCT systems and methods remain limited to stationary samples or samples moving much slower than the OCT frame rate for producing images devoid of detrimental motion artifacts.

One exemplary approach, although technologically challenging, can be to further increase the frame rate of OCT to capture rapid periodic vibrations. In order to accurately capture the motions of rapidly vibrating organs such as the middle ear ossicles and the tympanic membrane where the frequency of vibration can be as high as 20 kHz, it is necessary to capture at least 2 motion phases per cycle to accurately reconstruct the motion by Nyquist criterion, although compressional sensing algorithm may relax the requirement. The Nyquist sampling generally uses a maximum frame rate of about 40 kHz, which then translates to an A-line acquisition rate of more than 40 MHz. Such high acquisition rates not only pose a great technological challenge but results in a decrease in the signal-to-noise ratio (“SNR”), which likely eventually yields subpar images when compared to the ones taken with lower A-line rate systems. In addition, it may be necessary to capture more than 2 motion phases to avoid significant blurring of images, and this further increases the required frame and A-line rates to about 100 kHz and 100 MHz or beyond.

Another way of capturing rapid periodic motions is to sample a subset of a cycle over multiple cycles to recreate an illusion of slow motion. The basic principle is known as stroboscopy or time gated imaging [see Ref. 18]. In OCT systems and methods, some applications of gated imaging have been demonstrated with relatively slow time-domain OCT and Fourier-domain (swept source or spectral-domain) OCT procedures to image the embryonic hearts of a chicken and a mouse with the heartbeat frequency ranging from 1 to 10 Hz [see Refs. 19-21].

Accordingly, there may be a need to address at least some of the above-described deficiencies.

### SUMMARY OF EXEMPLARY EMBODIMENTS OF PRESENT DISCLOSURE

Thus, to address at least such issues and/or deficiencies, exemplary embodiments of system, method and computer-accessible medium for providing and/or utilizing optical coherence tomographic vibrography procedure(s) can be provided. For example, according to certain exemplary embodiments, such exemplary system, method and computer-accessible medium can be provided for OCT vibrography to capture snapshots of small-scale motion less than or equal to about 10 μm with vibration frequencies beyond frequencies of the typical A-line rates of Fourier-domain OCT, In addition, according to another exemplary embodiment of the present disclosure, exemplary system, method and computer-accessible medium can be provided to minimize and/or reduce a systematic and repeatable noise from the beam-scanning module by subtracting a separately recorded noise measurement from sample vibration information.

Certain exemplary embodiments of the present disclosure can be based on synchronization of beam scanning, data acquisition, and sample excitation signals in OCT. There are a number of the advantages of the exemplary embodiments over previously developed stroboscopic and gated imaging techniques. First, e.g., compared to prospective gated imaging where data can be selectively acquired in a pulsed manner [see Ref. 19], data acquisition can be continuous and therefore more time-efficient. Second, triggering allows the data acquisition to be synchronized with sample motion for accurate timing control. This exemplary procedure can be used so as to, e.g., ensures that the number of motion points resolved per cycle is approximately constant for all of the acquired cycles as long as motion is periodic and synchronized with the OCT data acquisition and scanning. Thus, e.g., many or all of the acquired motion phases can be provided in response to the excitation signals that ensures all of the spatial locations in the region of interest are experiencing the same phases of motion during acquisition. In short, triggering minimizes and even eliminates possible cycle-to-cycle time-misalignments in the snapshots reproduced. The previous retrospective gating techniques [see Refs. 20-21] are generally vulnerable to such misalignments because image acquisition is asynchronous with the sample motion. In summary, OCT vibrography procedures benefits from its unique ability to generate motion snapshots that are invulnerable to time-misalignments and increased speed in data acquisition compared to the previous gated techniques.

For example, The ability to quantify and visualize small-scale (typically, e.g., about 1 μm to 10 μm) oscillatory motions of objects in three-dimensions over a large bandwidth of frequencies (typically, e.g., about 1 Hz to 1 GHz) can have a wide range of application in acoustics, materials sciences and medicine. Capturing volumetric snapshots of periodic motion with optical coherence tomography is challenging when amplitudes are small and frequencies are high beyond several kHz. An exemplary OCT system according to exemplary embodiments of the present disclosure can be configured to obtain or capture such motions and provide volumetric “snapshots” that are reconstructed from the data acquired in synchrony with external stimulus applied to the objects. Such exemplary embodiments can have a broad range of applications from materials sciences to clinical diagnosis.

Accordingly, exemplary embodiments of apparatus, method and computer-accessible medium can be provided for obtaining image information regarding at least one portion of at least one sample. For example, using such exemplary embodiments, it is possible to use at least one arrangement to (i) receive or generate first data regarding a controlled physical excitation of the portion(s) and optical coherence second data associated with the sample(s). Further, it is possible, e.g., using such arrangement(s), to generate the image information based on the first data and the second data, wherein the controlled physical excitation is caused by a non-biological arrangement. The image information can include depth information within the portion(s).

In another exemplary embodiment, it is possible to use at least one further arrangement to cause the physical excitation of the at least one portion. The first data can be based on or used to cause the physical excitation. Such further arrangement(s) can comprises (i) a sound generating arrangement, (ii) an ultra-sound generating arrangement, (iii) a lead zirconate titnate (PZT) actuator arrangement, and/or (iv) a magnetic arrangement.

With still another exemplary arrangement, it is possible to acquire at least one first radiation from a reference and at least one second radiation of the sample(s), so as to generate the second data. The acquisition by synchronized with respect to the first data. With at least one additional arrangement, it is possible to forward at least one particular radiation to the sample(s) so as to scan the sample(s). Further, it is possible to control the scanning of the sample(s) based on the first data. The physical excitation can be at most about 10 μm in terms of an optical delay within the at least one portion. Further, the physical excitation can include a periodic signal.

According to yet another exemplary embodiment of the present disclosure, an amplitude of a response signals of the at least one portion to the physical excitation can be at most about 10 μm. It is also possible to generate the image information which includes a representative image of the portion(s) at a single instance in relative time with respect to the first data. The physical excitation can include a mechanical excitation. The image information can further include mechanical properties of the portion(s). The second data can be associated with a response to the physical excitation. The sample(s) can be an ear, and the image information can provide diagnostic information related to conductive hearing disorders and/or treatments. Further, the sample(s) can be an eye, and the image information can provide diagnostic information related to corneal disorders, cross-linking treatments, and/or refractive surgery.

These and other objects, features and advantages of the present disclosure will become apparent upon reading the following detailed description of exemplary embodiments of the present disclosure, when taken in conjunction with the appended drawings and enclosed claims.

### BRIEF DESCRIPTION OF THE DRAWINGS

Further objects, features and advantages of the present disclosure will become apparent from the following detailed description taken in conjunction with the accompanying drawings showing illustrative embodiments of the present invention, in which:

FIGS. 1A and 1B are block diagrams depicting components of exemplary embodiments of an OCT system and a system for synchronizing stimulus signals, data acquisition, and beam scan such as an OCT vibrography system according to the present disclosure;

FIGS. 2A-2D are graphical examples depicting different schematics of synchronization to cover a wide range of stimulus frequencies according to exemplary embodiments of the present disclosure;

FIG. 2E is a flow diagram for method of data processing to generate OCT vibrography images according to exemplary embodiments of the present disclosure;

FIGS. 3A-3C are exemplary graphs depicting measurements of scanner noise patterns cancellations and maximum motion sensitivity achieved with such exemplary embodiments of the present disclosure;

FIG. 4A is a block diagram depicting a system according to an exemplary embodiment of the present disclosure with a small drum stimulated with acoustic pressure;

FIGS. 4B and 4C are exemplary images generated with the exemplary system of FIG. 4A;

FIG. 5A-5H are exemplary images of chinchilla middle ear generated using OCT vibrography system, method and computer-accessible medium according to certain exemplary embodiments of the present disclosure;

FIGS. 6A-6E are exemplary images of chinchilla tympanic membrane generated with OCT vibrography system, method and computer-accessible medium according to certain exemplary embodiments of the present disclosure;

FIGS. 7a-7i are exemplary images of ossicular disorder models generated with OCT vibrography system, method and computer-accessible medium according to certain exemplary embodiments of the present disclosure;

FIG. 8 is a schematic diagram of an exemplary otoscope for OCT vibrography according to an exemplary embodiment of the present disclosure;

FIG. 9 is a block diagram of an exemplary OCT vibrography system for imaging vibrations corneal and crystalline lens with physical stimulus according to an exemplary embodiment of the present disclosure;

FIGS. 10A and 10B are exemplary functional vibrography images of a porcine cornea stimulated with acoustic stimulus generated using the exemplary system, method and computer-accessible medium according to further exemplary embodiments of the present disclosure;

FIG. 11A is a graph of an exemplary vibration magnitude of porcine corneas as a function of sound stimulus frequency for a pristine eye sample;

FIG. 11B is a graph of an exemplary vibration magnitude of porcine corneas as a function of sound stimulus frequency after treatment with collagenase;

FIG. 11C is a graph of an exemplary vibration magnitude of porcine corneas as a function of sound stimulus frequency after UV-induced riboflavin crosslinking procedure, respectively, measured according to further exemplary embodiments of the present disclosure; and

FIG. 12 is a block diagram of an OCT vibrography system for imaging vibrations of natural or synthesized materials for rheologic measurements according to yet further exemplary embodiment of the present disclosure.

Throughout the drawings, the same reference numerals and characters, if any and unless otherwise stated, are used to denote like features, elements, components, or portions of the illustrated embodiments. Moreover, while the subject disclosure will now be described in detail with reference to the drawings, it is done so in connection with the illustrative embodiments. It is intended that changes and modifications can be made to the described exemplary embodiments without departing from the true scope and spirit of the subject disclosure and appended claims.

### DETAILED DESCRIPTION OF EXEMPLARY EMBODIMENTS

According to exemplary embodiments of the present disclosure, technological advancements and crucial facilitating measurements can be used to perform OCT vibrography that can generate spatially resolved snapshots of rapid vibrations with small amplitudes much less than the axial resolution of OCT (e.g., approximately 10 μm). According to certain exemplary embodiments, OCT vibrography procedure(s) can be applied to diagnosing middle ear disorders and eye problems in clinical settings, as well as to the measurements of rheological properties of samples.

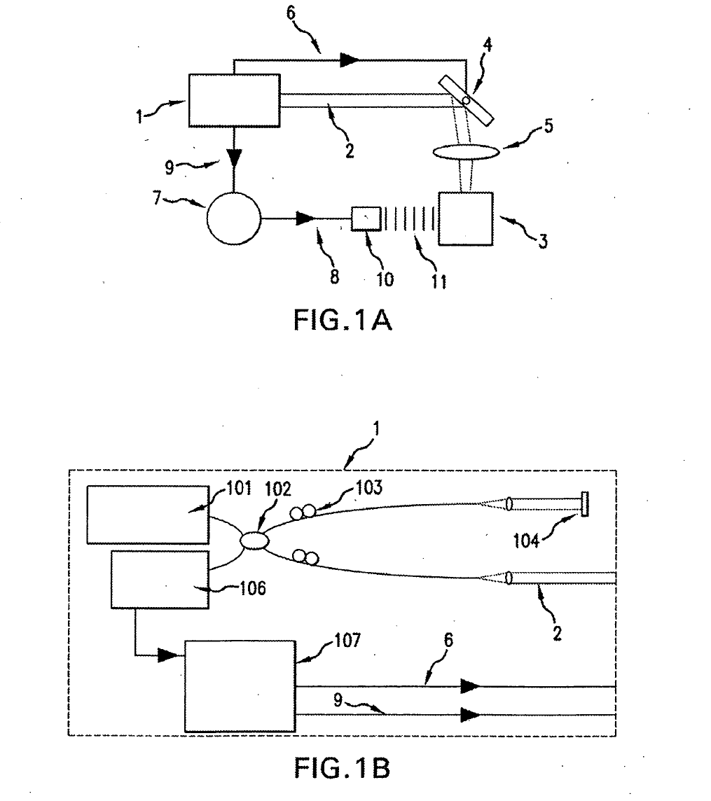

For signal generation and acquisition in OCT vibrography, motion excitation, OCT data acquisition, and beam scanning can be derived from the common time base for synchronization. FIG. 1A illustrates a block diagram of a system according to an exemplary embodiment of the present disclosure. This exemplary system includes at least one arrangement and/or apparatus 1 which can provide a first electromagnetic radiation 2 that can be delivered to a sample 3 or at least one portion thereof via at least one beam scanner 4 and/or an imaging lens 5. The exemplary arrangement/apparatus 1 can include at least one OCT system, e.g., such as a swept-source OCT system using wavelength swept lasers or spectral-domain OCT system using spectrometers. FIG. 1B shows an exemplary embodiment of a swept-source OCT system 1 which can include a wavelength swept laser 101, interferometer 102, polarization controlling module 103, at least one reference mirror 104, photodetectors 105, and digitizers 106 [see Ref. 14]. An exemplary spectral-domain OCT system uses a broadband source and a spectrometer with linescan cameras.

One exemplary form of the electromagnetic radiation 2 can include light in the visible or near infrared range. The electromagnetic radiation 2 for biological samples can have a wavelength between, e.g., about 450 nm and 1900 nm, although other wavelengths that can be safe for use in the specific sample are employable. The beam scanner(s) 4 can be or include a galvanometer-mounted mirror, MEMS mirror, PZT-based scanners [see Ref. 22], translation stages, or a spatial light modulator. The imaging lens 5 can be or include a spherical convex lens, graded index (GRIN) lens, aspheric lens, achromatic lens, objective lens, theta lens, axicon lens, Fresnel lens and/or cylindrical lens'for line focusing.

The common time base in the exemplary arrangement/apparatus 1 can control the scanner 4 with a synchronized scanner control signal 6. The synchronized scanner control signal 6 can be or include an electric signal, typically with one of the sinusoidal, triangular and saw-tooth waveforms. The waveform can include multiple discrete procedures. A synchronized excitation signal 8 can be directly generated from at least one arrangement 1 or an external signal generator 7 can be used to output the synchronized excitation signal with a synchronized function generator control signal 9 coming from the time base source in the arrangement 1. The synchronized function generator control signal 9 can be or include a digital transistor-transistor logic (TTL) signal or an analog signal. The synchronized excitation signal 8 can also be or include analog or digital signals.

The synchronized excitation signal 8 can be converted to a physical signal 11 that stimulates the sample through a transducer 10. The transducer 10 can be or include a loudspeaker, a mechanical transducer, an air pump, an ultrasonic probe, a PZT transducer, an electromagnet or a source capable of generating electromagnetic radiation either in continuous or pulsed waves. The physical signal(s) 11 can be and/or include acoustic pressure, pressure generated from electromagnetic radiation, magnetic pressure, ultrasonic pressure or contact based mechanical pressure.

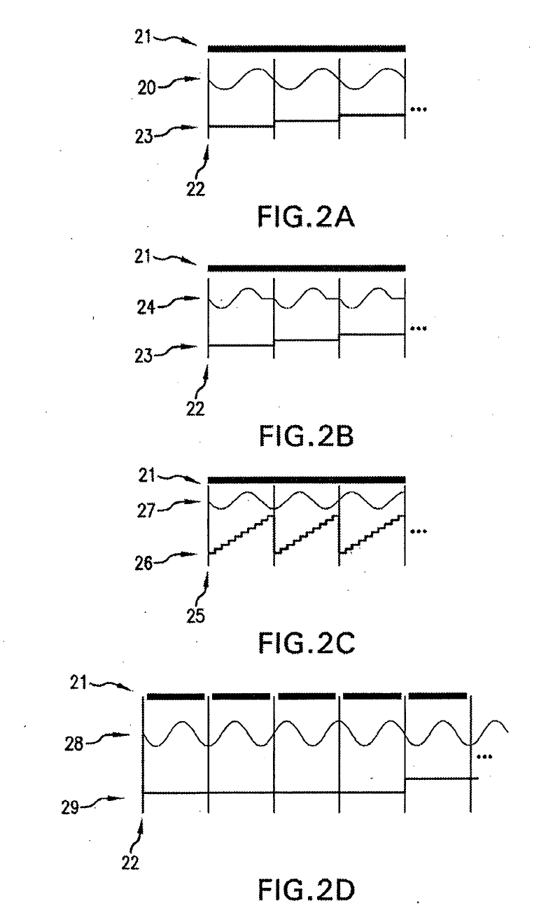

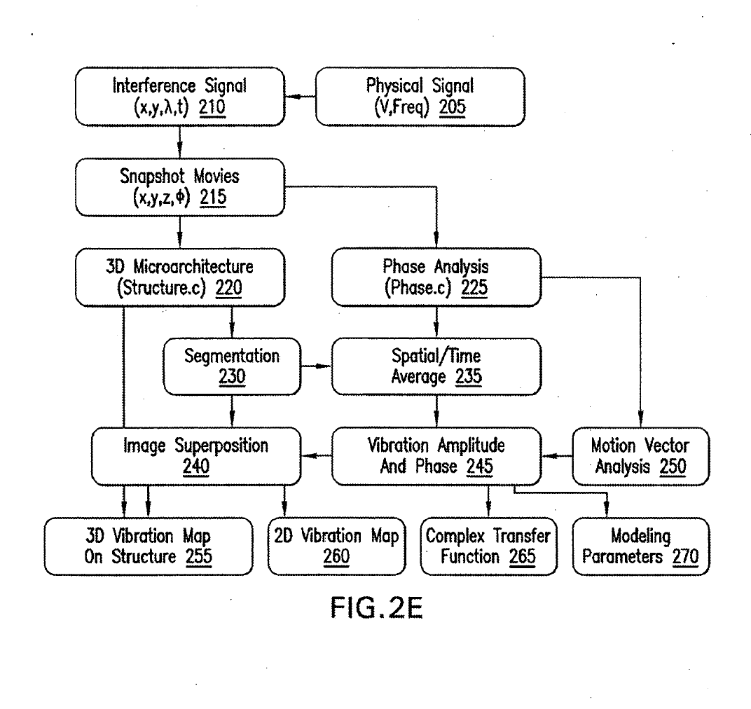

FIGS. 2A-2D illustrate graphical examples depicting different graphs of exemplary synchronization schemes to cover a wide range of stimulus frequencies according to exemplary embodiments of the present disclosure, including the exemplary synchronizations of various control and excitation signals. As shown in a first exemplary illustration of FIG. 2A, a frequency of an excitation signal 20 can be selected to be an integer fraction of the repetition frequency of A-lines 21 or vice versa. Triggering signals 22 can be generated at the completion of an integer number of cycles of the excitation signal 20 using an oscilloscope, a multi-function data acquisition card (DAQ), or a signal generator. A scanner signal 12 can be used to increment at the instance of the triggering signals 22. Throughout the exemplary procedure associated with FIG. 2A, an integer number of A-lines can be acquired per cycle. It is also possible to generate the excitation signals 20 from the triggering signals 22 where an exact integer number of vibration cycles are generated between the triggering signals 22. In the second exemplary illustration presented in FIG. 2B, excitation signal 24 may not have to be an integer fraction of the repetition frequency of A-lines 21. Therefore, the remaining cycle can include, but not be limited to, a DC signal. An exact integer number of A-lines 21 can be acquired during an integer number of sample vibration cycles to eliminate quantization error in data acquisition. In a first exemplary mode of operation, the beam scanner is steered to the next lateral location after a user-defined number of vibration cycles, capturing full vibration cycles at each location. When the scan is completed over the user-defined region, the A-line profiles acquired at the same phase of oscillation can be grouped together to produce “snapshot” vibrographs of the sample.

FIG. 2C shows a third exemplary illustration of the synchronization scheme according to an exemplary embodiment of the present disclosure. For example, as illustrated in FIG. 2C, a start trigger signal 25 can mark the end of each beam scan signal 26. The start trigger signal 25 can be derived from the beam scan signal 26, or vice versa. In this exemplary configuration associated with FIG. 2C, the excitation signal 27 and the beam scan signal 26 can be synchronized such that the phase the excitation signal in the next scan marked by the next start trigger signal 25 is advanced and/or retarded by an integer fraction of 2π, which represents the total number of A-line signals acquired each step. The stepping of phase can be achieved by, e.g., tuning the frequency of the signal and outputting both the excitation and scan signals on the same cue from the start trigger signal 25. It is also possible to trigger the phase stepping of the excitation signal 27 by an integer fraction of 2π at each instance of the start trigger signal 25. The number of discrete procedures within each period of the beam scan signal 26 is set equal to the number of A-lines acquired within that period. The A-line profiles at each location are further interpolated and the A-line profiles corresponding to the same phase of oscillation are grouped together to produce snapshot vibrographs of the sample undergoing forced vibration.

When the vibration frequency of excitation signals exceeds the A-line rate of an OCT system, another exemplary mode of synchronization associated with FIG. 2D can be used. Each A-line 21 can include, e.g., a wavelength sweep over the bandwidth of few tens and hundreds of nanometers. During synchronized data acquisition, every A-line signal can be digitized by the DAQ to a finer unit of wavelength that is given by the inverse of the DAQ maximum digitization rate. Using a similar principle to FIG. 2C, the phase of an excitation signal 28 is advanced or retarded by an integer fraction of 2π at the start trigger signal 22 of each A-line acquisition. A beam-scan signal 29 can increment each procedure at the completion of the 2π phase change in the excitation signal 28. Wavelength profiles within each A-line that correspond the to the same phase of oscillation can be grouped together to form A-line snapshot profiles. Additionally, further grouping of the A-line snapshots with respect to the phase of oscillation can provide exemplary snapshot vibrographs. Exemplary synchronization modes presented in FIGS. 2A-2C can be, e.g., limited by the A-line rate (MHz regime), while the mode shown in FIG. 2D can be limited by the DAQ sampling rate (GHz regime). The total image acquisition time for mode illustrated in FIGS. 2A and 2B can be given by N number of locations scanned in X and Y dimensions divided by the excitation signal frequency. The total image acquisition time for modes in FIGS. 2C and 2D can be equivalent to N number of locations divided by A-line rate and multiplied by the number of total phase procedures.

FIG. 2E illustrates a flow diagram of exemplary image processing procedure(s) according to exemplary embodiments of the present disclosure. First, the OCT interference signal can be acquired as a function of the beam coordinate in X and Y, wavelength λ, and time t (procedure 210). The physical excitation data, such as the voltage amplitude V and frequency, are also provided (i.e., from procedure 205). Snapshot data: Time-resolved complex parameters of signal amplitude and phase can be calculated via Fourier analysis of the OCT interference signal and excitation data (procedure 215). The resulting information can be rearranged to generate 3D snapshots at different phases φ across the vibratory cycle (procedure 220). From the snapshot movie, microstructural data and phase data can be obtained. 3D micro-architecture: The magnitude of the scattering signal is used to produce time-independent three-dimensional magnitude scattering datasets. The data are often presented in log scale. Segmentation can be performed to identify and present regions of interest (procedure 230).

Phase analysis (motion)—procedure 225: The OCT derived optical phase angle, φ, can be expressed as, e.g., φ(t)=Δφ sin(2πft)+φₙ, where Δφ=4π/λ*δz is the optical phase amplitude corresponding to the amplitude of motion δz, λ is the center wavelength of the swept laser, f is the vibration frequency, and φₙ is the intrinsic phase noise given by <δφₙ>²=1/(2*SNR), where SNR is the signal-to-noise ratio in the intensity of the interference signal. This exemplary phase angle can be extracted from the complex raw data with Fourier analysis and is subsequently rearranged to give snapshot images. For example, each phase resolved snapshot image can have the format of (x, y, z, δz(φ)). These exemplary datasets can he used to derive displacement (nm) of the sample in the z-dimension (parallel to the imaging beam).

Spatial/Time average—procedure 235: Both segmented structural data and motion data can be averaged spatially and temporally. Spatiotemporal averaging of the motion data can increases the motion sensitivity by N^{1/2}, where N is the number of spatially or temporally averaged pixels.

Image superposition—procedure 240: The displacement data can be mapped onto the structural images at each phase in the motion cycle. Vibration amplitude and phase—procedure 245: Volumetric vibration amplitude and phase can be determined and/or calculated from, e.g., the Fourier analysis superposed 4D dataset (x, y, z, δz(φ)). The amplitude and phase maps can provide an exemplary motion vector analysis (phase map—procedure 250) and complex transfer function (both amplitude and phase—procedure 265) used as parameters for modeling in procedure 270. The structural and phase data can be superimposed to generate 2D cross-sectional and volumetric vibrography images—procedures 255 and 260. Other parameters, such as complex transfer function and motion vector, can he extracted as well from the superimposed data.

For tracking movements that are within the range of 1 to 10 microns, it is possible to utilize pixel-by-pixel registration or tracking method. Imagine a point spread function (PSF) in either axial or lateral dimension. Displacements much smaller than the full-width-at-half-maximum (FWHM) of the PSF can be detected by tracking the pixels that make up the PSF. It is possible to track as small as few microns using this method given a high signal-to-noise ratio (SNR) from the sample. For applications that prefer a higher precision for a detection of sub-micron displacements, phase sensitive OCT procedures can be employed. For image registrations in OCT, amplitudes of the light reflected from the sample can be used to render traditional 2-D cross-sectional images. The maximum motion sensitivity of the exemplary OCT system that can be achieved with the amplitude information of reflected light is on the other of several microns using the pixel-by-pixel registration. By observing the changes in phase, however, it is possible to sense displacements much smaller than the axial resolution of the OCT system, as small as a sub-nanometer scale. Unlike wide-illumination stroboscopic techniques [see Ref. 5], OCT vibrography procedures can be used to acquire data over many more vibration cycles at multiple lateral locations. Therefore, the exemplary OCT vibrography procedures can be applicable to samples in stable oscillation and slow macroscopic motion during the scan duration. The low frequency macroscopic motions can be spectrally separated from the high frequency vibrations in the acoustic range, and the acquired vibrography signals can be high pass filtered to reduce the motion artifacts from macroscopic motions.

As shown in FIG. 2E and indicated herein above, the physical signals can be provided. Then, the intensity and phase of the interference signals from a specific location in the external and internal surface of the sample can be measured during the oscillatory motion [see Ref. 23]. The phase angle, φ, can be expressed as: φ(t)=Δφ sin(2πft)+φₙ, where Δφ=4π/λ*δz is the phase amplitude corresponding to the vibration amplitude δz, f is the vibration frequency, and φₙ is the intrinsic phase noise given by <δφₙ>²=1/(2*SNR), where SNR is the signal-to-noise ratio in the intensity of the interference signal. Averaging of N measurements where N is the number of cycles or spatial locations acquired during a single vibration cycle can improve the amplitude sensitivity. The amplitude sensitivity is given by λ/4π*(2*SNR*N)^{1/2}.

Exemplary OCT vibrography systems and procedures can achieve a sub-nanometer amplitude sensitivity (˜10⁻¹¹ m) by minimizing and/or reducing mechanical and ambient acoustic noise. It is possible to achieve synchronization among data acquisition, beam scanning, data acquisition, and sample actuation by generating all the control signals from the internal time-base clock of the DAQ board using the illustration of FIG. 2A. Following mode 1, the frequency of the driving sinusoidal waveform (S) was set at an integer fraction of the A-line rate, so that an integer number, N_{A}, of A-lines were acquired during each cycle of sample oscillation. The beam scan can be synchronized to the oscillation so that after a predetermined number, N_{B}, of cycles, the beam was stepped to the next lateral location using the galvanometer beam scanner (B). The exemplary lowest possible N_{A} and N_{B} are 2 and 1 respectively, where two motion phases are captured per cycle (Nyquist limit) and the beam is moved after the completion of each vibration cycle. FIG. 2A illustrates an exemplary situation for N_{A}=10 and N_{B}=1. When the scans over the regions of interest in the sample were completed, A-line profiles acquired at the same phase of the oscillation (φ=0, 2π/N_{A}, . . . or 2π(N_{A}−1)/N_{A}) can be grouped to produce “snapshots” or vibrographs of the sample [see Ref. 24].

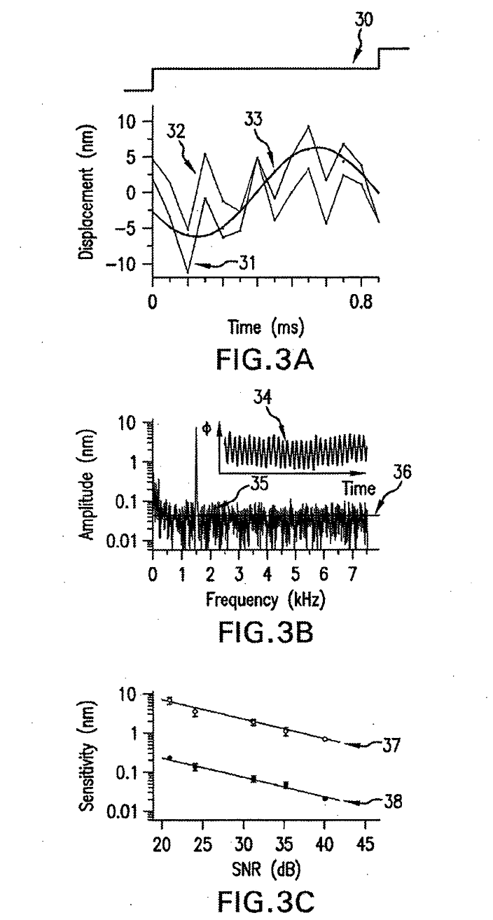

In certain applications which utilize exemplary OCT procedures and systems, at least one scanner-mounted mirror can be used to steer the imaging beam to scan the region of interest on a sample. Types of signals applied for scanner operation can include the saw-tooth waveform, sine waveform, and triangular waveform. As shown in FIG. 3A, the signals can comprise of discrete step(s) 30. The duration of each procedure can be given by the inverse of A-line rate and the imaging OCT beam is parked at a specific location during each exemplary procedure. The scanners can have a unique finite procedure response 32 that can result in a noisy measurement 31 taken with phase sensitive OCT. This deterministic noise can be numerically canceled out by subtracting a separately recorded trace of its step response 32 measured from a stationary sample. The resulting corrected measurement 33 shows a noise free sinusoidal motion of the sample.

To test the sensitivity of the exemplary system, it is possible to image a sample vibrating at about 1.5 kHz with an A-line rate of 15 kHz using the exemplary synchronization scheme associated with the graph of FIG. 2A. Such exemplary scheme can provide an integer number of A-lines (10) per vibration with a single cycle of vibration per galvanometer step. The amplitude of vibration can be measured to be about 7 nm, as confirmed with laser Doppler velocimetry. It is possible to measure the phase angle of the OCT pixel corresponding to the top surface of the glass plate where the SNR of the pixel was 30 dB. The inset shown in the graph of FIG. 3B illustrates an exemplary time trace 34 of phase angle. The spectrum 35 shown in FIG. 3B illustrates a Fourier domain trace of the measured phase angles (N=2880), showing a sample vibration peak at about 1.5 kHz and a noise floor of about 46 pm, e.g., close to the theoretical limit 36 of 42 pm. SNR was varied using a graded neutral density filter to explore the relationship between SNR and sensitivity. FIG. 3C shows the exemplary vibration sensitivity measured at SNR levels ranging from 20 to 40 dB for single-cycle (N=1) 37 and multi-cycle averaging (N=1000) 38. In both cases, SNR-limited theoretical sensitivity can be obtained. The maximum sensitivity at SNR=40 and N=1000 (integration time is 1/1.5 s) can be about 26 pm.

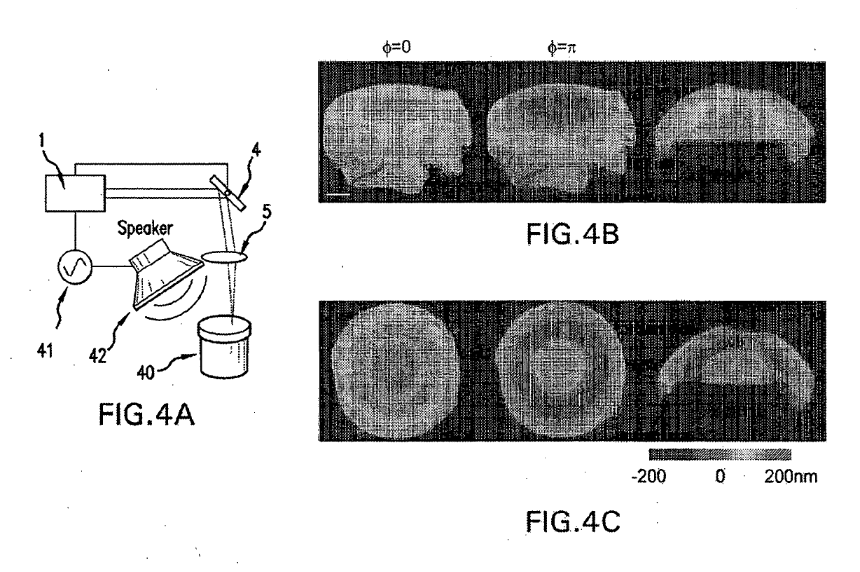

It is possible to apply the exemplary OCT vibrography system to capture 3D snapshots of an acoustically-driven drum head consisting of a 200 micron thick latex membrane stretched over and glued (or otherwise connected) to a 5 mm diameter metal tube 40 (as shown in FIG. 4A). Sinusoidal signals from a function generator 41 can be applied to a loudspeaker 42 and the radiated sound was used to evoke vibration of the drumhead. The OCT beam can be scanned over the entire membrane, covering 512 by 256 XY spatial points. By sweeping the sound frequency, it is possible to locate a resonance at about 800 Hz for the fundamental resonance mode, (0,1) mode, where the maximum displacement was observed.

FIG. 4B shows exemplary snapshots of the membrane taken at two opposite vibration phases, φ=0 and π, respectively, at f=800 Hz (the number of A-lines was 20 per cycle and A-line rate can be about 16 kHz). The cutaway view reveals the homogeneous vibration across the full thickness of the latex membrane. The vibration snapshots show the Bessel profile of the vibration mode with a 3D spatial resolution of about 10 μm and a motion-phase resolution of 2π/20. The total acquisition time can be about 163.8 s to acquire the full 3D data set (e.g., 512 by 256 by 400 pixels in XYZ and 20 motion phases). For example, decreasing the image volume and increasing the frequency of excitation signals can reduce the total acquisition time. With high frequency excitation signals of about 10 kHz, the total scan time can be about 13 seconds, while maintaining the vibration sensitivity and image volume.

As the sound frequency is increased, higher order vibration modes can be obtained. FIG. 4C shows exemplary snapshots of the motion at about 1.78 kHz (the number of A-lines can be 8), when the resonant excitation of the second-order radial mode, (0, 2) mode, became evident. The ratio of the resonance frequencies between the first and second radial modes can be close to 2.3 as expected from the acoustic theory [see Ref. 25]. A three-dimensional contour plot can highlight the characteristic amplitude pattern of the second order radial mode. The snapshots can illustrate the exemplary profile of the vibration mode with a motion-phase resolution of about 2π/9. The total data acquisition time can be about 73.6 s.

An exemplary embodiment of OCT vibrography system and method according to the present disclosure can be used in the field of otology, where controlled small-scale rapid periodic motions are involved. Among 36 million (about 17 percent) American adults suffering from hearing loss, conductive hearing loss due to middle-ear disorders constitutes a large proportion secondary to sensorineural hearing loss [see Refs. 26, 27]. Accurate diagnosis of middle-ear diseases can be important to effective and timely treatments of hearing loss. Current clinical diagnostic tests such as tympanometry, otoscopy, and LDV are limited to the surface measurement of the tympanic membrane (TM) and ossicular disorders are not generally visible through the intact TM [see Refs. 28, 29]. Thus, there is a need for accurate and objective diagnosis of middle ear function in air-filled ears in which the TM is intact.

One exemplary application of OCT vibrography procedures according to the present disclosure can assess ossicular structure and the sound-induced motion of the TM and ossicles through the intact TM with unprecedented sensitivity to nanometer vibrations. The exemplary OCT vibrography procedures and system can be tested with fresh cadaveric chinchilla heads since chinchilla is a widely used model in hearing research [see Refs. 30, 31].

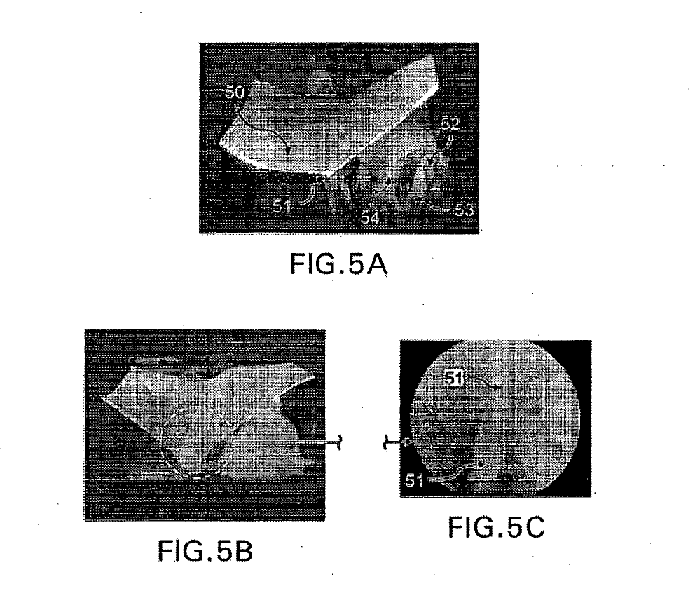

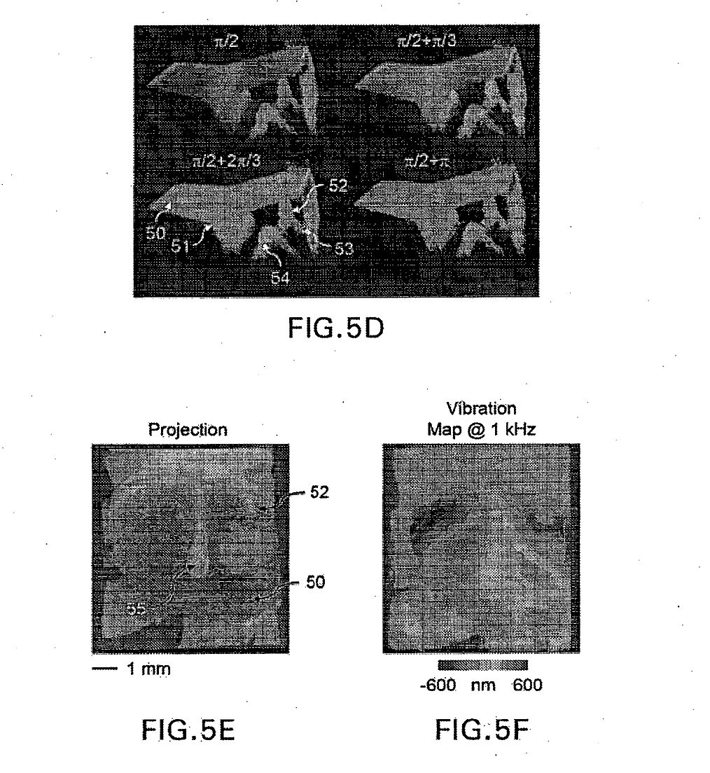

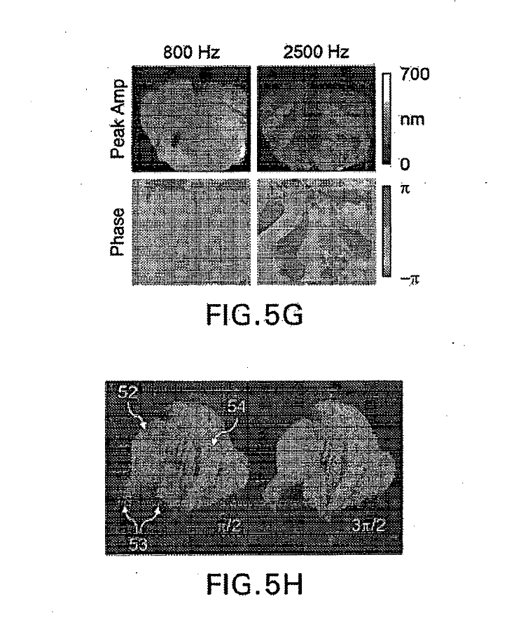

FIG. 5A shows an exemplary structural image of a chinchilla ear from the superior. Anatomical features such as the TM 50, manubrium 51, distal incus 52, and stapes 53, cochlea 54, and umbo 55—the umbo is the central attachment of the TM to the malleus—are identified from the structural image. FIG. 5B shows an exemplary image of the top half of the TM and ossicles from the inferior. FIG. 5C illustrates a lateral view of the TM surface. The manubrium of the malleus attached the TM appears to draw the TM toward the tympanic cavity. In FIG. 5D, an exemplary sequence of the reconstructed vibrography images (color encoded 300 to 100 nm from blue to red) is shown at several different phases of motion at about 500 Hz of stimulus frequency. The stapes and distal incus can be seen to move with uniform amplitude and phase. As common in open-cavity experiments, the wall of the middle ear cavity also vibrates with small but measurable amplitude. FIG. 5E shows an exemplary static structural projection image of a different chinchilla ear. An exemplary color-encoded OCT vibrography snapshot image (pi/4 motion phase) of the chinchilla middle ear at about 1 kHz is shown in FIG. 5F. The exemplary peak-hold amplitude map (top) and vibration phase map (bottom) at two different frequencies are shown in FIG. 5G. The phase map shows that the entire TM moves in phase at 800 Hz whereas more complex higher-order resonances appear at about 2500 Hz. FIG. 5H shows exemplary color-encoded OCT vibrography images of the chinchilla incus and stapes at about 1 kHz at two opposite motion phases. The exemplary data provide strong evidence for our unprecedented capability of simultaneous imaging the shape and motion of the ossicles together with those of the TM.

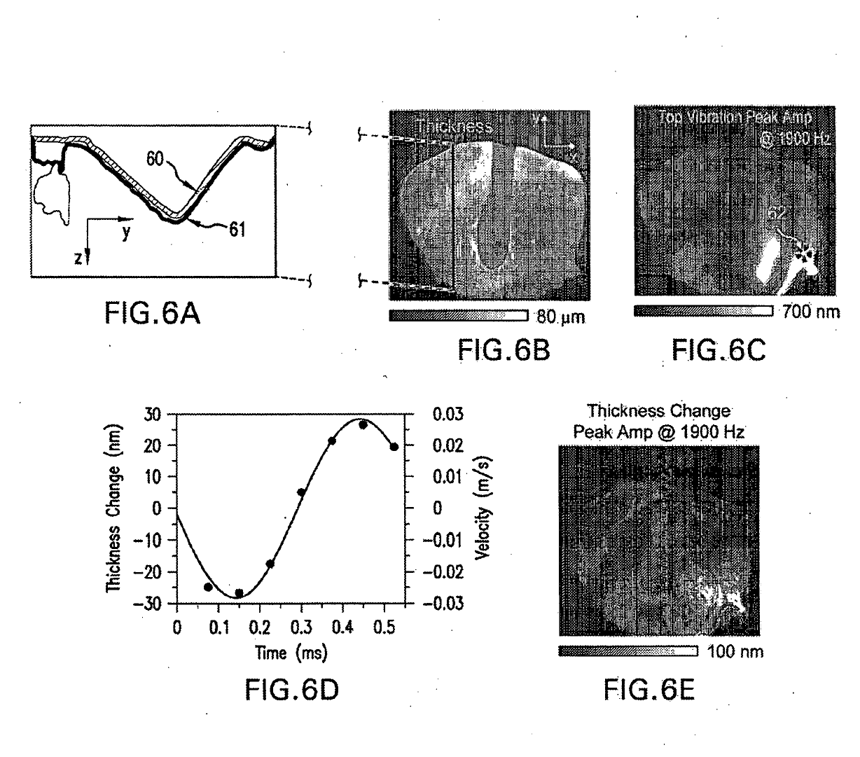

Using the exemplary OCT's unique access to the subsurface vibration the thickness of the TM and its changes during -the sound-induced displacement- can be measured. For example, it is possible to first use a segmentation procedure to trace the top 60 and bottom surfaces 61 of the TM (FIG. 6A). Then, the thickness of the TM can be estimated by measuring the distance between the top and bottom layers of the TM (see FIG. 6B). FIG. 6C shows the exemplary peak-hold vibration amplitude map of the top surface at about 2 kHz and 110 dB SPL. The exemplary variation in vibration amplitude and phase between the top and bottom pixels during sound stimulation defines the subtle changes of thickness. The ratio of changes in thickness to the original thickness can be related to the local tensile strain. Since a velocity map can be obtained from the vibrography images, it is possible to determine the spatially resolved viscoelastic moduli of the TM from the ratio of the velocity to the local strain. FIG. 6D shows both a thickness change and a velocity of the region of interest on TM (e.g., see dotted circle in FIG. 6C) and FIG. 6E shows the exemplary peak-hold thickness change amplitude map of the TM at about 2 kHz and 110 dB SPL.

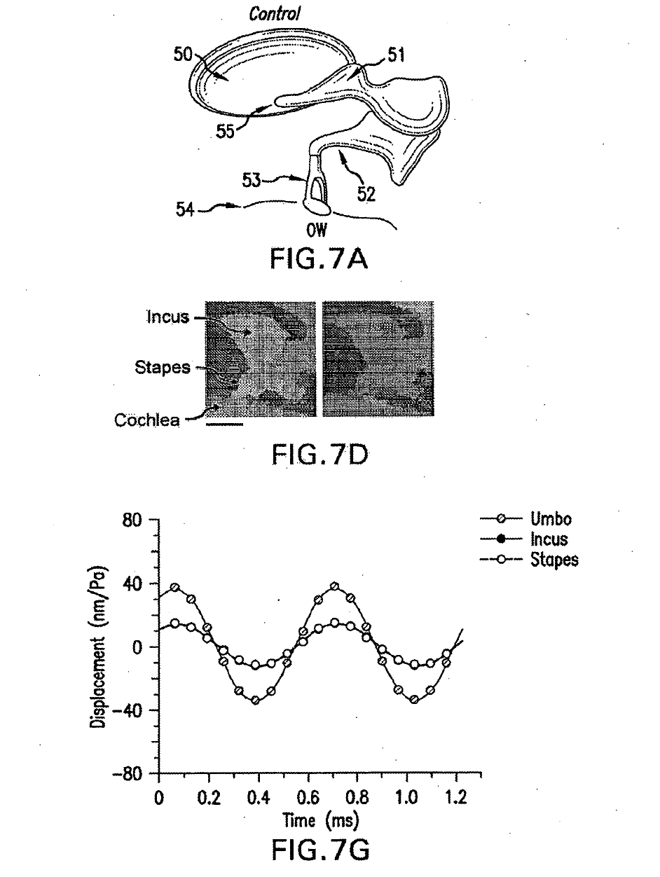

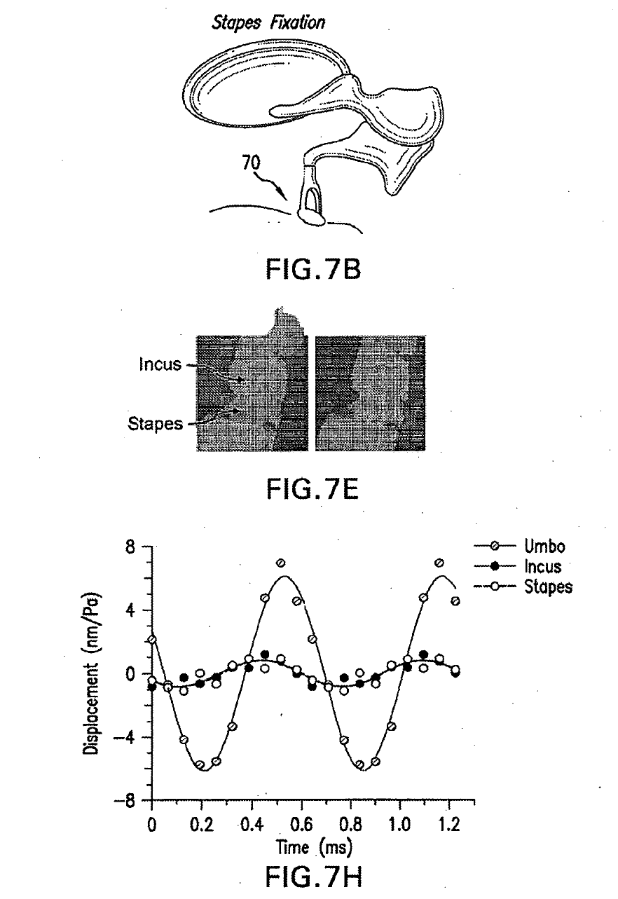

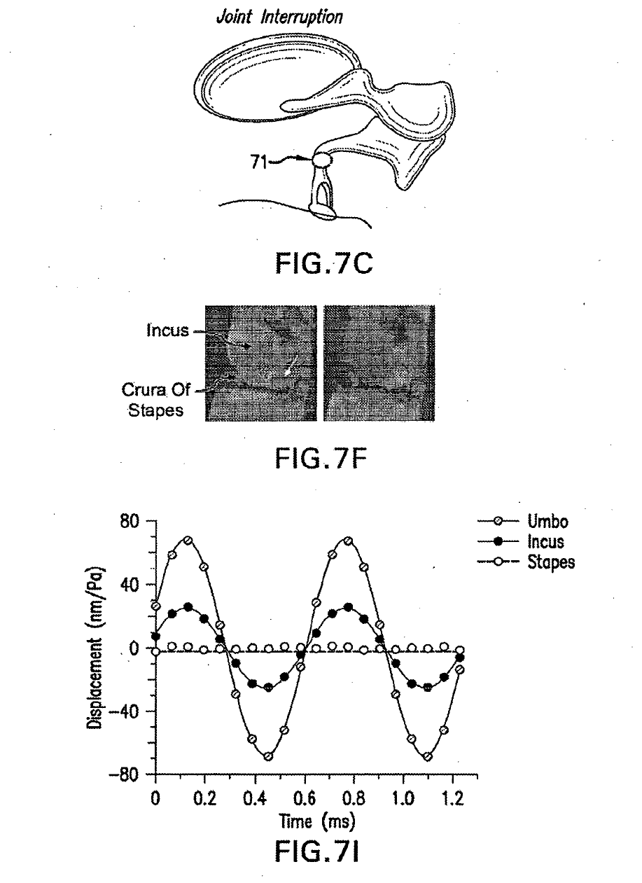

For an exemplary preliminary evaluation, it is possible to apply an exemplary OCT vibrography procedure to image to a chinchilla model of middle-ear disorders. These exemplary models [see Ref. 32] simulate otosclerosis by immobilizing the stapes footplate with glue 70 and the interruption of the I-S joint by a surgical manipulation 71 (see, e.g., FIGS. 7a-7c, top panels). When a sound wave at about 1.5 kHz and 104 dB SPL can be applied before and after these manipulations, the exemplary vibrography images of the TM showed noticeable but small differences between the control and diseased states. In contrast, the vibrography images can indicate dramatic changes in the ossicular motions. In the normal state, both the incus and stapes can be seen to move in phase at similar amplitudes of 15 nm/Pa (1 Pa=94 dB SPL), about 40% of the movement of the umbo (see, e.g., FIGS. 7d and 7g). After the stapes fixation, the vibrations of all three ossicles were attenuated by a factor of 8-20 in amplitude (see, e.g., FIGS. 7e and 7h). The residual stapes motion can be seen to precede that of the umbo by about 100-110 μs. When the I-S joint is broken, a small vibration of the stapes was detected as anticipated. However, the vibration amplitudes of incus and umbo actually increased by a factor of about 1.5 can be compared to the control (see, e.g., FIGS. 7f and 7i). An increase can be attributed to a decrease in the mechanical load on the malleus and incus after the I-S joint interruption. Exemplary preliminary data support our hypothesis that OCT vibrography can detect the changes in amplitudes and phases of motions at various locations in the ossicular chain through the intact TM, providing useful diagnostic information about ossicular pathologies. In the human ear, the line of sight of the stapes and incus can be better since the stapes and incus are positioned more toward the middle of the TM providing a less obstructed view compared to the chinchilla model.

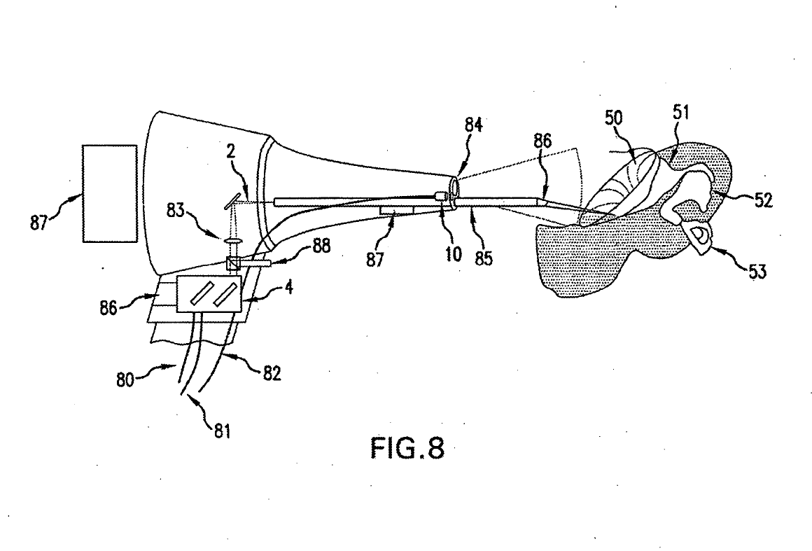

For an exemplary clinical application, an exemplary hand-held OCT vibrography otoscope can be used. FIG. 8 shows an schematic diagram of such hand-held OCT otoscope according to an exemplary embodiment of the present disclosure. With the previous demonstration of an optical fiber in the standard otoscope for axial low-coherence interferometry [see Ref. 33], the exemplary configuration can consist of three key components in the body of an otoscope. Exemplary components can include but are not limited to an optical fiber 80, wires 81 to control the scanning module 4, a coupling lens 83 to focus the electromagnetic radiation 2, the transducer 10 capable of generating physical signals to excite the sample, a wire 82 to deliver driving signals to the transducer 10, an illuminator 84 with visible electromagnetic radiation, and a graded index (GRIN) rod lens 85. An angled prism mirror 86 can he attached at the end of the GRIN lens 85. The mirror 86 can have either an aluminum-coated or gold-coated surface the angle is configurable between about 30 and 90 degrees. The electromagnetic radiation 2 can be provided into the GRIN probe 85 through the scanner 4 and the coupling, lens 83. The GRIN probe 85 can be mounted in the otoscope speculum so that, when the speculum is in place in the ear canal, the probe tip is adjusted at an optimal distance and angle with respect to the TM. Synchronized mechanical translation systems 85 and 86 can be inserted in the otoscope to translate the scanner 4 and the GRIN probe 85 simultaneously to adapt the instrument to different subjects with varying ear canal lengths. When the electromagnetic radiation 2 used for OCT procedures and/or systems are outside the range for human vision and a detector 87, it is possible to employ an additional visible aiming electromagnetic radiation 88 collinear to the imaging electromagnetic radiation 2. The detector 87 can be a CMOS camera, a CCD camera with silicone, InGaAs or extended InGaAs.

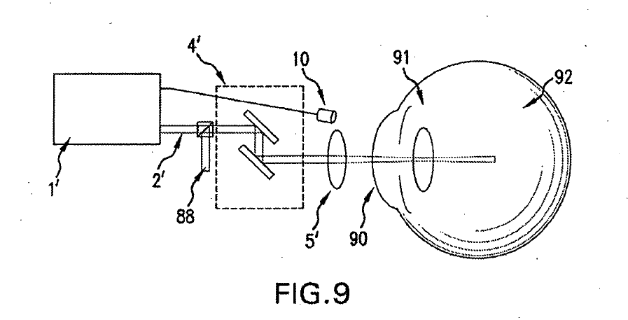

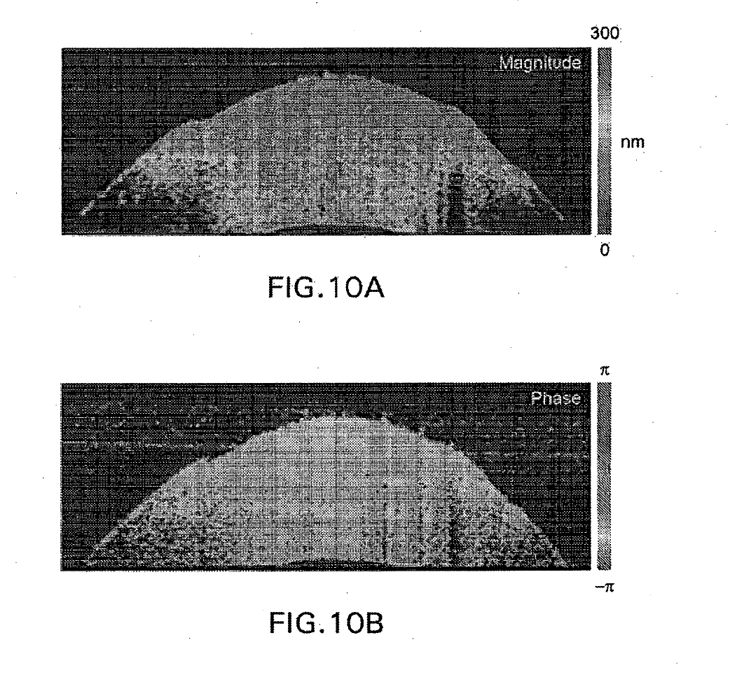

Another exemplary application area of OCT vibrography is diagnosis in ophthalmology, especially in the analysis of mechanical properties of cornea and crystalline lens. Structural scanning of retina and the anterior segment with OCT is a well-established procedure in ophthalmology [see Refs. 12-13]. Prevalent ocular problems such as cornea ectasia, cataracts, and presbyopia (loss of lens accommodation) have been affected by the degrading qualities of the cornea and lens either with age or pathologies. An exemplary arrangement and/or configuration to measure the elastic properties of the cornea and lens in situ and noninvasively can assist with an early prospective diagnosis, pre-surgical and/or post-surgical assessment. An exemplary embodiment for ophthalmic applications is illustrated in FIG. 9, in which acoustic or ultrasonic pressure waves can excite the cornea, and OCT vibrography images the cornea 90, lens 91, and retina 92. At least one exemplary arrangement 1′ can generate at least one electromagnetic radiation 2′. A scanner 4′ can control the radiation 2′ which can be focused with a focusing lens 5′ onto a sample 90 (e.g., the eye). A physical transducer 10 can be used to stimulate the sample. A visible aiming electromagnetic radiation arrangement can also be used. Representative exemplary information, such as, e.g., vibration amplitude, phase maps of the porcine cornea at 1 kHz of acoustic stimulus are shown in FIGS. 10A and 10B.

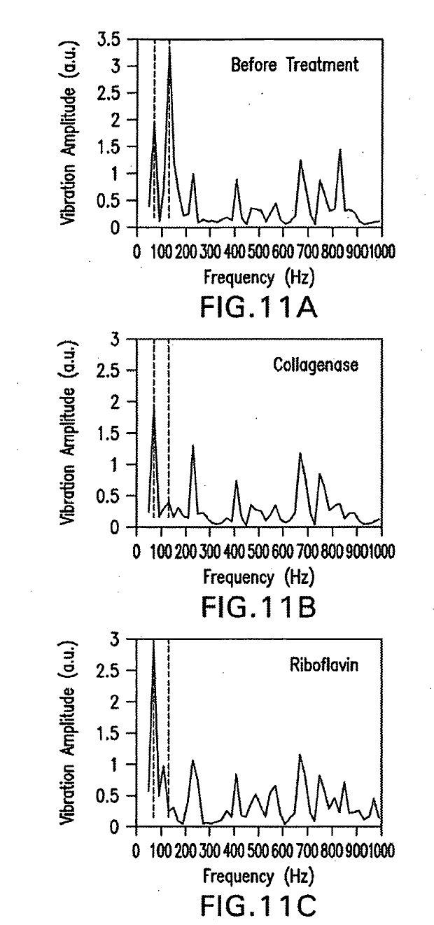

Exemplary information that can be obtained using the exemplary apparatus can include the mechanical resonance spectrum of the eye. FIG. 11A shows a graph of a nominal peak vibration amplitude of the cornea surface measured as a function of the acoustic frequency. Several distinct mechanical resonance peaks of the cornea are shown (two of the lowest order resonance peaks are marked by dotted lines). After applying collagenase to the cornea, noticeable differences in the typical resonance spectrum can be detected (as shown in FIG. 11B). After riboflavin cross-linking, there were significant changes in terms of acoustic resonance frequencies and relative amplitudes of different resonance peaks (as shown in FIG. 11C). Such resonance information may be used as a measure of the mechanical properties of the corneal tissues and can be useful for the diagnosis of keratoconus or for monitoring of treatment effects.

Another exemplary embodiment of OCT vibrography system and method according to the present disclosure can be applied in the field of rheometry and microrheology. Current state-of-the art technologies for rheometry include quasi-static stress-strain axial measurement, Dynamic Mechanical Analyzer, dynamic light scattering, and ultrasound pulse echo technique. Such techniques apply cyclic stress to the specimen ranging from DC (quasi-static measurement) up to 10 MHz (ultrasound pulse echo technique) and measure torsional angle, axial displacement or ultrasound time of flight. Such techniques either provide 2D surface information, e.g., lacking the depth profile of strain or a low-resolution single line profiles from the time of flight measurements along single axis. Without the depth profile or high spatial resolution, material characterization can be limited to homogeneous materials that do not allow layered or sophisticated structures of tissue and wave propagations on the surface level. In contrast, exemplary OCT vibrography procedure can provide 3D information that can allow characterization of more complicated tissue structure that consists of multiple layers with high spatial resolution.

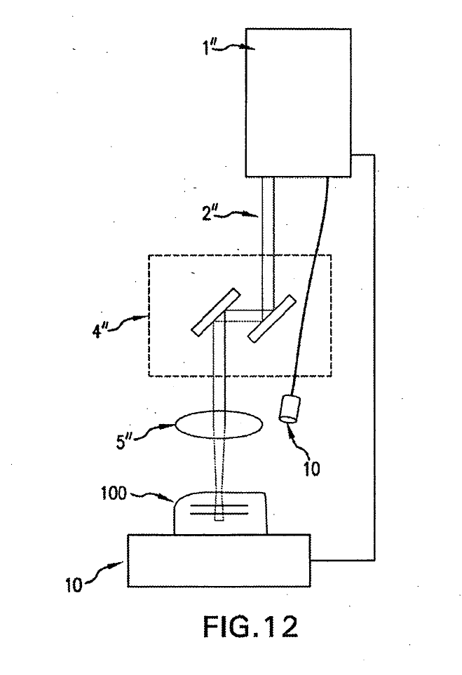

As shown in FIG. 12, the exemplary arrangement 1″ can generate at least one electromagnetic radiation 2″, can be is controlled with a beam scanner 4″, and focused onto the sample 100 with a focusing module 5″. The sample 100 can be a thick or thin sample with multiple heterogeneous or homogeneous layers. The sample 100 can be transparent, highly scattering, or translucent. OCT vibrography can cover a broad range of cyclic strain up to few megahertz generated from an actuator including PZT, speaker, and ultrasonic transducers, allowing a further characterization of elastic modulus as a function of excitation frequency. This feature can ultimately allow a further insight in power-law relation over a broad range of frequencies. Moreover, this power-law relation can provide a link between existing rheological techniques and a recently developed technique called 3D Brillouin confocal microscopy that can measure the elastic properties of samples in the frequency regime in the gigahertz (GHz) range [see Ref. 34]. Since the exemplary OCT vibrography system and/or method is capable of characterization in microscopic dimensions, only small amounts of material are needed, being able to increase the microscopic understanding of complicated materials. Furthermore, the exemplary OCT vibrography system and/or method can be used in micro-rheology where characterization of cells or cellular cytoskeletons such as actins and microtubules is crucial. More biologically relevant longitudinal mode of compression (osmotic compression) as opposed to the transverse or shear modes can be characterized.

The foregoing merely illustrates the principles of the disclosure. Various modifications and alterations to the described embodiments will be apparent to those skilled in the art in view of the teachings herein. Indeed, the arrangements, systems and methods according to the exemplary embodiments of the present disclosure can be used with and/or implement any OCT system, OFDI system, SD-OCT system or other imaging systems, and for example with those described in International Patent Application PCT/US2004/029148, filed Sep. 8, 2004 which published as International Patent Publication No. WO 2005/047813 on May 26, 2005, U.S. patent application Ser. No. 11/266,779, filed Nov. 2, 2005 which published as U.S. Patent Publication No. 2006/0093276 on May 4, 2006, and U.S. patent application Ser. No. 10/501,276, filed Jul. 9, 2004 which published as U.S. Patent Publication No. 2005/0018201 on Jan. 27, 2005, and U.S. Patent Publication No. 2002/0122246, published on May 9, 2002, the disclosures of which are incorporated by reference herein in their entireties. It will thus be appreciated that those skilled in the art will be able to devise numerous systems, arrangements and methods which, although not explicitly shown or described herein, embody the principles of the disclosure and are thus within the spirit and scope of the present disclosure. It should be understood that the exemplary procedures described herein can be stored on any computer accessible medium, including a hard drive, RAM, ROM, removable disks, CD-ROM, memory sticks, etc., and executed by a processing arrangement and/or computing arrangement which can be and/or include a hardware processors, microprocessor, mini, macro, mainframe, etc., including a plurality and/or combination thereof. In addition, certain terms used in the present disclosure, including the specification, drawings and claims thereof, can be used synonymously in certain instances, including, but not limited to, e.g., data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it can be explicitly incorporated herein in its entirety. All publications referenced herein can be incorporated herein by reference in their entireties.

### REFERENCES

1. D. Kleckner and D. Bouwmeester, “Sub-kelvin optical cooling of a micromechanical resonator,” Nature 444, 75-78 (2006).
2. T. J. Kippenberg and K. J. Vahala, “Cavity opto-mechanics,” Opt. Express 15, 17172-17205 (2007).
3. T. P. Burg, M. Godin, S. M. Knudsen, W. Shen, G. Carlson, J. S. Foster, K. Babcock, and S. R. Manalis, “Weighing of biomolecules, single cells and single nanoparticles in fluid,” Nature 446, 1066-1069 (2007).
4. M. H. Xu and L. H. V. Wang, “Photoacoustic imaging in biomedicine,” Rev. Sci. Instrum. 77(2006).
5. J. T. Cheng, A. A. Aarnisalo, E. Harrington, M. D. Hernandez-Montes, C. Furlong, S. N. Merchant, and J. J. Rosowski, “Motion of the surface of the human tympanic membrane measured with stroboscopic holography,” Hearing Research 263, 66-77 (2010).
6. X. Liang, M. Orescanin, K. S. Toohey, M. F. Insana, and S. A. Boppart, “Acoustomotive optical coherence elastography for measuring material mechanical properties,” Opt. Lett. 34, 2894-2896 (2009).
7. B. F. Kennedy, X. Liang, S. G. Adie, D. K. Gerstmann, B. C. Quirk, S. A. Boppart, and D. D. Sampson, “In vivo three-dimensional optical coherence elastography,” Optics Express 19, 6623-6634 (2011).
8. R. K. Chhetri, K. A. Kozek, A. C. Johnston-Peck, J. B. Tracy, and A. L. Oldenburg, “Imaging three-dimensional rotational diffusion of plasmon resonant gold nanorods using polarization-sensitive optical coherence tomography,” Phys. Rev. E 83, 040903 (2011).
9. R. K. Wang and A. L. Nuttall, “Phase-sensitive optical coherence tomography imaging of the tissue motion within the organ of Corti at a subnanometer scale: a preliminary study,” Journal of Biomedical Optics 15, 056005 (2010).
10. M. A. Choma, A. K. Ellerbee, C. H. Yang, T. L. Creazzo, and J. A. Izatt, “Spectral-domain phase microscopy,” Opt. Lett. 30, 1162-1164 (2005).
11. C. Joo, T. Akkin, B. Cense, B. H. Park, and J. E. de Boer, “Spectral-domain optical coherence phase microscopy for quantitative phase-contrast imaging,” Opt. Lett. 30, 2131-2133 (2005).
12. M. R. Hee, J. A. Izatt, E. A. Swanson, D. Huang, J. S. Schuman, C. P. Lin, C. A. Puliafito, and J. G. Fujimoto, “Optical coherence tomography of the human retina,” Arch. Ophtalmol. 113(3), 325-332 (1995).
13. C. A. Puliafito, M. R. Flee, C. P. Lin, E. Reichel, J. S. Schuman, J. S. Duker, J. A. Izatt, E. A. Swanson, and J. G. Fujimoto, “Imaging of macular diseases with optical coherence tomography,” Ophthalmology, 102(2):217-29 (1995).
14. S. H. Yun et al, “Comprehensive volumetric optical microscopy in vivo,” Nat. Med. 12, 1429-1433 (2006).
15. G. J. Tearney, S. Waxman, M. Shishkov, B. J. Vakoc, M. J. Suter, M. I. Frilich, A. E. Desjardins, W. Y. Oh, L. A. Bartlett, M. Rosenberg, and B. E. Bouma, “Three-dimensional coronary artery microscopy by intracoronary optical frequency domain imaging,” J. Am. Coll.Cardiol.Img. 1:752-61 (2008).
16. W. Y. Oh, S. H. Yun, G. J. Tearney, and B. E. Bouma, “115 kHz tuning repetition rate ultrahigh-speed wavelength-swept semiconductor laser,” Opt. Lett. 30(23):3159-61 (2005).
17. W. Wieser, B. R. Biedermann, T. Klein, C. M. Eigenwillig and R. Huber, “ Multi-Megahertz OCT: High quality 3D imaging at 20 million A-scans and 4.5 GVoxels per second,” Opt. Express 18, 14685-14704 (2010).
18. E. G. Amparo, C. B. Higgins, D. Farmer, G. Gamsu and M. McNamara, “Gated MRI of cardiac and paracardiac masses: initial experience,” Am. Journal of Roent. 143, 1151-1156 (1984).
19. M. W. Jenkins et al, “4D embryonic cardiography using gated optical coherence tomography,” Opt. Express 14, 736-748 (2007).
20. S. Yazdanfar, M. D. Kulkarni and J. A. Izatt, “High resolution imaging of in vivo cardiac dynamics using color Doppler optical coherence tomography,” Opt. Express 1, 424-431 (1997).
21. A. Mariampillai et al, “Doppler optical cardiogram gated 2D color flow imaging at 1000 fps and 4D in vivo visualization of embryonic heart at 45 fps on a swept source OCT system,” Opt. Express 15, 1627-1638 (2007).
22. T. H. Tsai et al, “Piezoelectric-transducer-based miniature catheter for ultrahigh-speed endoscopic optical coherence tomography,” Biomed. Opt. Express 2, 2438-2448 (2011)
23. B. J. Vakoc, S. H. Yun, J. F. de Boer, G. J. Tearney, and B. E. Bouma, “Phase-resolved optical frequency domain imaging,” Opt. Express 13, 5483-5493 (2005).
24. E. W. Chang, J. B. Kohler, and S. H. Yun, “Triggered optical coherence tomography for capturing rapid periodic motion,” Scientific Reports 1 (2011).
25. R. S. Christian, R. E. Davis, A. Tubis, C. A. Anderson, R. I. Mills, and T. D. Rossing, “Effects of air loading on timpani membrane vibrations,” J. Acous. Soc. Am. 76, 1336-1345 (1984).
26. National Institute of Deafness and Other Communication Disorder, http://www.nidcd.nih.gov/health/statistics/Pages/quick.aspx
27. M. C. Holley, “The auditory system, hearing loss and potential targets for drug development,” Drug Discov. Today 10, 1269-1282 (2005).
28. J. Jerger, “Impedance terminology,” Arch. Otolaryngol. Head Neck. Surg. 101, 589-590. (1975)
29. C. H. Chein et al, “Prediction of the Pure-Tone Average from the Speech Reception and Auditory Brainstem Response Thresholds in a Geriatric Population,” ORL-J Oto-Rhino-Laryngol. Relat. Spec. 70, 366-372 (2008)
30. G. D. Ehrlich et al, “Mucosal biofilm formation on middle-ear mucosa in the chinchilla model of otitis media,” J. Am. Med. Assoc. 287, 1710-1715 (2002)
31. P. A. Vrettakos, S. P. Dear and J. C. Saunders, “Middle-ear structure in the Chinchilla—a quantitative study,” Am. J. Otolaryng. 9, 58-67 (1988)
32. H. H. Nakajima, M. E. Ravicz, J. J. Rosowski, W. T. Peake and S. N. Merchant, “Experimental and clinical studies of malleus fixation,” Laryngoscope 115, 147-154 (2005).
33. C. T. Nguyen, H. H. Tu, E. J. Chaney, C. N. Stewart and S. A. Boppart, “Non-invasive optical interferometry for the assessment of biofilm growth in the middle ear,” Biomcd. Opt. Express 1, 1104-1116 (2010)
34. G. Scarcelli, S. H. Yun, “Confocal Brillouin microscopy for three-dimensional mechanical imaging,” Nat. Photon. 2, 39-43 (2008)

1. An apparatus for obtaining image information regarding at least one portion of at least one sample, comprising:
at least one computer arrangement configured to (i) receive or generate first data regarding a controlled physical excitation of the at least one portion and optical coherence second data associated with the at least one sample, and (ii) generate the image information based on the first data and the second data, wherein the controlled physical excitation is caused by a non-biological arrangement, and wherein the image information includes depth information within the at least one portion.

2. The apparatus according to claim 1, further comprising at least one further arrangement which is configured to cause the physical excitation of the at least one portion.

3. The apparatus according to claim 2, wherein the first data is based on or used to cause the physical excitation.

4. The apparatus according to claim 2, wherein the at least one further arrangement comprises at least one (i) a sound generating arrangement, (ii) an ultra-sound generating arrangement, (iii) a lead zirconate titnate (PZT) actuator arrangement, or (iv) a magnetic arrangement.

5. The apparatus according to claim 1, further comprising at least one further arrangement which is configured to acquire at least one first radiation from a reference and at least one second radiation of the at least one sample, so as to generate the second data, wherein the acquisition is synchronized by the at least one arrangement with respect to the first data.

6. The apparatus according to claim 1, further comprising at least one additional arrangement which is configured to forward at least one particular radiation to the at least one sample so as to scan the at least one sample, wherein the at least one arrangement is further configured to control the scanning of the at least one sample based on the first data.

7. The apparatus according to claim 1, wherein the physical excitation is at most about 10 μm in terms of an optical delay within the at least one portion.

8. The apparatus according to claim 1, wherein the physical excitation includes a periodic signal.

9. The apparatus according to claim 1, wherein an amplitude of a response signals of the at least one portion to the physical excitation is at most about 10 μm.

10. The apparatus according to claim 1, wherein the at least one arrangement generates the image information which includes a representative image of the at least one portion at a single instance in relative time with respect to the first data.

11. The apparatus according to claim 1, wherein the physical excitation includes a mechanical excitation.

12. The apparatus according to claim 1, wherein the image information further includes mechanical properties of the at least one portion.

13. The apparatus according to claim 1, wherein the second data is associated with a response to the physical excitation.

14. The apparatus according to claim 1, wherein the at least one sample is an ear, and wherein the image information provides diagnostic information related to at least one of conductive hearing disorders and/or treatments.

15. The apparatus according to claim 1, wherein the least one sample is an eye, and wherein the image information provides diagnostic information related to at least one of corneal disorders, cross-linking treatments, or refractive surgery.

16. A method for obtaining image information regarding at least one portion of at least one sample, comprising:
receiving or generating first data regarding a controlled physical excitation of the at least one portion and optical coherence second data associated with the at least one sample; and
generating the image information based on the first data and the second data, wherein the controlled physical excitation is caused by a non-biological arrangement, and wherein the image information includes depth information within the at least one portion.

17-30. (canceled)

31. A non-transitory computer-accessible medium which includes executable instructions, wherein, when the executable instructions are executed by a computing arrangement, the computer arrangement is configured to execute procedures comprising:
receiving or generating first data regarding a controlled physical excitation of the at least one portion and optical coherence second data associated with the at least one sample; and
generating the image information based on the first data and the second data, wherein the controlled physical excitation is caused by a non-biological arrangement, and wherein the image information includes depth information within the at least one portion.
